(19) United States
(12) Patent Application Publication
(10) Pub. No.: US 2009/0214677 A1
(43) Pub. Date: Aug. 27, 2009

(54) Pharmaceutical Composition Containing an Extract of a Solidago Species

(75) Inventors: Péter Literati Nagy, Budapest (HU); Kalman Tory, Budapest (HU); Attila Kolonics, Budapest (HU); Agnes Keri, Budapest (HU); Lajos Laszlo, Budapest (HU); Laszlo Jaszlits, Budapest (HU); Agnes Bajza, Budapest (HU); Sandor Bernath, Telki (HU); Laszlo Vigh, Szeged (HU); Tibor Bodnar, Budapest (HU); Janos Egri, Budapest (HU)

Correspondence Address: K.F. ROSS P.C., 5683 RIVERDALE AVENUE, SUITE 203 BOX 900, BRONX, NY, 10471-0900, US

(73) Assignee: ELSO MAGYAR BIODROG KUTATÓ ÉS FEJLESZTO KFT, Biatorbagy (HU)

(21) Appl. No.: 11/991,362

(22) PCT Filed: Aug. 30, 2006
(86) PCT No.: PCT/HU2006/000070

§ 371 Date: Mar. 25, 2009

(30) Foreign Application Priority Data
Sep. 1, 2005 (HU) P 0500814

(51) Int. Cl. A61K 36/28; A61P 25/28

(52) U.S. Cl. 424/725

(57) ABSTRACT

An extract of a part of a Solidago species, wherein said part has grown above the earth, or the solid residue remaining after the removal of the solvent content of the extract as the active ingredient is used for the regeneration of the organism of a mammal after long-lasting immobilization, anorexia, states following a disease or accompanied by weight loss as well as for muscle development or muscle growth during muscle developing training, treatment of muscular strain and adaptation to high-altitude as well as for the prevention or treatment of neurodegenerative diseases and/or motility disorders of the gastrointestinal system.

### FIELD OF THE INVENTION

The invention refers to uses of an extract of a goldenrod (Solidago) species. More particularly, the invention refers to the use of an extract of a part of a Solidago species, wherein said part has grown above the earth, or the solid residue remaining after the removal of the solvent content of the extract as the active ingredient for increasing the mitochondrial genesis as well as for the prevention and/or treatment of diseases due to damages of the mitochondrion or a reduced function of the enzyme constitutive nitric oxide synthase.

### BACKGROUND OF THE INVENTION

Damages of the mitochondrion plays an important role in the formation of several diseases, while other diseases are developed owing to disturbances in the constitutive nitric oxide synthase system.

The mitochondrion is an essential organelle of the cell which occurs in varying number in the cytoplasm of every cell. That is the site of the cell's energy production. 98% of the oxygen used by the human organism is applied by the mitochondria for energy production. Oxidative phosphorylation taking place in the mitochondrion produces a considerable amount of ATP (adenosine triphosphate) that stores the energy needed by the cell. Thus, the number and state of mitochondria is determinative from the point of view of life.

In function of physical requirement, the oxidative capacity of the striated muscle is able to change by an order of magnitude. The myofibrillar protein type of the muscle is changed and the mitochondrion content of the muscle is increased during accommodation to the load. In the regulation of mitochondrial function and formation, the transcription factor PGC-1α of the coactivator PPARγ (peroxisome proliferator-activated receptor γ) has key role. Mitochondrial biogenesis is also influenced by the calcium/calmoduline dependant kinase IV (CaMKIV), calcineurine, AMP-kinase [Zong H et al.: AMP kinase is required for mitochondrial biogenesis in skeletal muscle in response to chronic energy deprivation, Proc. Natl. Acad. Sci., 99, 15983 (2002)], MEF2 (myocyte enhancer factor 2), p38 MAPK as well as CREB, however, their effect is produced mainly through PGC-1α [Nisoli E. et al.: Mitochondrial biogenesis as a cellular signaling framework, Biochemical Pharmacology 67, 1 (2004.)]. CAMKIV and calcineurin have an indirect influence on the activity of the promoter of PGC-1α, while p38 MAPK exerts its effect through the phosphorylation of PGC-1α and delaying the effect of the endogenic inhibiting domain [Fan M. et al., Genes & Development, 18, 278 (2004)]. According to recent observations, the nitric oxide produced by the endothelial nitric oxide synthase enzyme—through the increase of the activity of the guanilate cyclase enzyme and the cGMP level—plays a fundamental part in inducing the expression of PGC-1α and, thus, in the regulation of mitochondrial genesis [Nisoli, E.: Mitochondrial biogenesis in mammals. The role of endogenous nitric oxide. Science, 299, 896 (2003)].

In addition to the energy production, the mitochondrion takes part also in the regulation of other physiological processes, for example, it plays a role in the regulation of the insulin secretion of β-cells, in the oxygen perception of the pulmonary vessels as well as the sinus caroticus. The mitochondrion contains the key enzymes that define the rate of steroid biosynthesis and the carbonic acid anhydrase enzyme that is essential for the secretion of gastric acid. The mitochondrion has a great part in the regulation of calcium signalization through the uptake of cytosolic calcium [Gunter T. E. et al.: Mitochondrial calcium transport: mechanism and functions, Cell Calcium, 28, 285 (2000)]. The heat generation ability of the brown adipose tissue is based on the detachment of oxidative phosphorylation, however, this process is only of secondary importance in man. The mitochondrion is of key importance in the regulation of the programmed cell death (apoptosis) [Martinou J. C., Green D. R.: Breaking the mitochondrial barrier, Nat. Rev. Mol. Cell. Biol., 2, 63 (2001)].

The damage of mitochondrion is the cause of several diseases. A specific mutation of mitochondrial DNA results in the development of type I or insulin-dependent diabetes mellitus [Maassen J. A. et al.: Mitochondrial diabetes: molecular mechanisms and clinical presentation, Diabetes, 53 Suppl 1, 103 (2004)]. In the type II or noninsulin-dependent diabetes mellitus, the basic disorder that starts the patomechanism consists in a reduced sensitivity of the tissues against insulin i.e. insulin resistance. According to recent examinations, a reduced oxidative phosphorylation capacity of the mitochondria can be in the background of insulin resistance [Petersen K. F. et al.: Mitochondrial dysfunction in the elderly: possible role in insulin resistance, Science 300, 1140; Petersen K. F. et al.: Impaired mitochondrial activity in the insulin resistant offspring of patients with type II diabetes, N. Engl J. Med., 350, 665 (2004)]. A genetic relation between the PGC-1α gene playing a key role in the regulation of mitochondrion function and mitochondrion biogenesis on the one hand, and obesity and diabetes on the other hand was shown in Danish and Japanese population [Ek, J. et al., Diabetologia, 44, 2220 (2001); Hara et al., Diabetologia, 45, 740 (2002)]. Furthermore, reduced levels of PGC-1α were detected in patients suffering from type II diabetes mellitus [Patti, M. et al.: Coordinated reduction of genes of oxidative metabolism in humans with insulin resistance and diabetes: Potential role of PGC1 and NRF1, PNAS, 100, 8466 (2003)].

Several chronic neurodegenerative diseases such as Parkinson's disease, Alzheimer's disease, Huntington's disease and ALS (amyotrophic lateral sclerosis) are accompanied by damaged mitochondrial function. It is deemed that the damage of mitochondria contributes to the progression of the disease [Scon E. A., Manfredi G., J. Clin. Invest., 111, 303 (2003)]. In these chronic neurodegenerative diseases, a change of conformation of certain neuronal proteins can be shown, wherein said change is accompanied by the function alteration and aggregation of the proteins. Partly, through the enhanced formation of free radicals, the mitochondrial dysfunction has a causal role in inducing the change of protein conformation, partly, the damaged mitochondrion itself becomes a target of the metabolic cascade induced by the change of protein conformation, thus, contributing to the progression of the disease. The mitochondrial dysfunction results in the destruction of nerve cells primarily through enhanced free radical formation, reduced energy generation, disorder of calcium homeostasis and endoplasmatic reticulum.

The study of the lifetime of animals revealed that a long lifetime is coupled with a low level of reactive oxygen species (ROS) [Perez-Campo R. et al.: The rate of free radical production as a determinant of the rate of aging: evidence from the comparative approach, J. Comp. Physiol., 168, 149 (1998)]. According to the mostly accepted aging theories, the process of aging is related to the oxidative damage [Hekimi S., Guarente L.: Genetics and specificity of aging process, Science, 299, 1351 (2003)]. The cumulation of mitochondrial defects and the enhanced formation of free radicals are considered as a cause in the development of diseases related to aging [Fridovich, I.: Mitochondria: are they the seat of senescence? Aging Cell, 3, 13 (2004)]. The role of mitochondrion in the aging process is supported by the fact that the point of attack of the genes that enhance lifetime is either the mitochondrion itself or the antioxidant mechanisms closely connected with the mitochondrion.

The functioning of the constitutive nitric oxide synthase system and its role in the patomechanism of diseases is outlined as follows.

Nitric oxide (NO) is a ubiquitous signal transducer molecule having very significant regulatory roles. Nitric oxide has an important role in the vasodilation through the relaxation of the smooth musculation of vessels. The aggregation and activation of blood platelets as well as the proliferation of the smooth muscle cells of vessel are inhibited by nitric oxide that plays a role also in the regulation of heart muscle contraction and relaxation. Nitric oxide is essential in the regulation of the motility of the gastrointestinal tract, primarily through inhibiting the contraction of the sphincters e.g. pylorus (or pyloric sphincter) [Huang P. L., Am. J. Cardiol., 82, 57S (1998); Takahashi T., J. Gastroenterol., 38, 421 (2003)].

Nitric oxide is produced from L-arginine by at least three different enzymes [neuronal nitric oxide synthase (nNOS, NOS1), inducible nitric oxide synthase (iNOS, NOS2) and endothelial nitric oxide synthase (eNOS, NOS3)].

Neuronal type nitric oxide synthase is predominantly expressed in specific neurons of the brain, in non-adrenergic, non-cholinergic autonomic nerve cells, in muscles and in the macula densa region of the renal tubules, however, it is present at lower level in many other tissues as well. In the activation of nNOS enzyme, elevation of intracellular Ca⁺⁺ concentration and protein phosphorylation plays an immediate role. Furthermore, recent observations have revealed that the alteration of the expression level of the enzyme has a significant effect on the regulation of the activity thereof, too [Sasaki, M. et al., Proc. Natl. Acad. Sci. USA, 97, 8617 (2000)].

The examination of nNOS knockout animals revealed a series of disease conditions where impaired nNOS enzyme function had significant role in the pathogenesis [Mashimo, H., Am. J. Physiol., 277, 745 (1999)]. The proper motility of the whole gastrointestinal tract, especially the relaxation of sphincters, depends on the activation of nNOS in non-adrenergic, non-cholinergic neurons [Takahashi, T., J. Gastroenterol., 38, 421 (2003)]. Nitric oxide produced by the nNOS enzyme regulates the muscle tone of the sphincter in the lower esophagus, pylorus, anus and the sphincter of Oddi through the inhibition of contraction. The diminished relaxation of sphincters disturbs the function, in this way e.g. the insufficient relaxation of pylorus (or pyloric sphincter) disturbs the coordinated mechanism of gastric emptying. For example, in nNOS knockout mice, gastric dilatation and stasis develop due to the long evacuation of the stomach. The level and function of nNOS expression is severely damaged in both type I and type II diabetes with the consequence that a dysfunction of the gastrointestinal system occurs in about 75% of the patients. Diabetic gastropathy syndrome is characterized by prolonged gastric emptying, flatulence, nausea, vomiting, abdominal pain that deteriorate life quality [Koch K. L., Dig. Dis. Sci., 44, 1061 (1999)]. Insulin treatment that restores nNOS expression and NO level [Watkins C., J. Clin. Invest., 106, 373 (2000)] or supplementing NO through NO donors eliminates the diabetic gastrointestinal motility disturbance, thus, supporting the pathological role of NO in the disease.

A consequence of insufficient nNOS enzyme activity in the sphincter of Oddi is the syndrome of “lazy gall bladder”. Owing to the lack of nitric oxide due to nNOS, the relaxation of the sphincter of Oddi is not sufficient resulting in an inhibited flow of bile from the gall bladder which leads to digestive troubles due to acholia as well as to cholecystectasia and cholestasis. The consequence of the latter symptoms is an enhanced risk of inflammatory diseases and formation of gallstones. Since bile plays an essential role in the lipid metabolism, the reduced bile secretion results in higher cholesterol level in the blood which can contribute to the development of metabolic syndrome [JAMA, 285, 2486 (2001)].

Other gastrointestinal motility and function disturbances in the pathomechanism of which the reduced nNOS function may have significant role include achalasia, hypertrophic pylorus stenosis, Hirschprung's disease, functional digestion disorders, ileus and colitis. Significant therapeutical effect can be expected in these diseases by restoring the nNOS function. Also in simple hypermotility disorders, favourable effects can be awaited through the enhancement of the nNOS function and the restoration of the local neuronal reflexes.

In a similar way, the enzyme plays a fundemental role in the erection of penis [Cuevas A. J. et al, Biochem. Biophys. Res. Commun., 312, 1202], therefore, the unsufficient activity of nNOS enzyme, mainly as a consequence of diabetes, is a frequent cause of erectile disfunctions.

The nNOS enzyme activity has an essential role in normal muscle function. Recent data indicate that in certain muscle degenerations, for example in Duchene muscular distrophy, also the function of nNOS enzyme is damaged [S. Froehner, Trends in Molecular Medicine, 8, 51 (2002)]. Restoration of the inadequate nNOS function improved the symptoms of the disease in animals.

The unsufficient function of nNOS enzyme can be responsible also for diseases related to aggressive behaviour since animal studies indicate that diminished expression and function of the enzyme result in serotonin dysfunction (descreased serotonin turnover, deficient serotonin receptor function) leading to aggressive behaviour [Chiavegatto, S. et al., Proc. Natl. Acad. Sci. USA, 98, 1277 (2001)]. It is believed that deficient nNOS function has a role in disease patterns related to aggressive behaviour and certain disturbances of sexual attitude.

Nitric oxide may have both pro- and anti-apoptic effect. Based on experimental observations, the suitable activity of the enzyme is essential in different nerve regeneration processes e.g. for recovery in traumatic peripheral nerve lesion [Keilhoff, G. Et al., Cell. Mol. Biol., 49, 885 (2003)]. Nitric oxide donors can be useful in the inhibition or treatment of arteriosclerosis [Herman, A. G. és Moncada, S.: Therapeutic potential of nitric oxide donors in the prevention and treatment of athero-sclerosis, Eur. Heart J., 2005 May 25].

The enzymes nNOS and eNOS are jointly called as constitutive nitric oxide synthase (cNOS) enzyme.

As a summary, it can be stated that mitochondrial damage as well as the reduced function of constitutive nitric oxide synthase enzyme may develop various diseases. Although some of the diseases can be treated with available synthetic drugs, however a drawback of synthetic drugs resides in the side-effects, often highly disagreeable unwanted effects thereof. In addition, there is no drug presently available that could restore the activity of the nNOS enzyme in different tissues or could achieve mitochondrial genesis.

The aim of the invention is the prevention or treatment of diseases related with mitochondrial damage and/or a reduced function of cNOS enzyme by a pharmaceutical composition based on a medicinal herb extract.

Various Solidago species have been used in European phytotherapy for more than 700 years mainly in urulogical and antiphlogistic, wound-healing compositions. Even the name of the plant indicates the application field: the Latin word “solidare” has a meaning of confirmation, healing. The sprout of the plant collected at the beginning of flowering is known under the name Virgae-aureae herba or Consolidae sarracenicae, while the root of the plant collected in autumn or in the early spring is known under the name Virgae-aureae radix or Consolidae sarracenicae radix. The European Scientific Cooperative on Phytotherapy (ESCOP) issued a monography on a Solidago species namely Solidaginis virgaureae herba already in 1996 [ESCOP MONOGRAPHS on the medicinal uses of plant drugs/Solidaginis virgaureae herba, 1996]. The prescriptions referring to the medicinal herb have been accepted according to the French Pharmacopoeia based on which only the inflorescence part of the sprout can be used as medicinal herb [Pharmacopée Française, X^{e} édition, Solidage—Solidago virga-aurea, Adrapharm, Paris, 1982]. In the German Pharmacopoeia (DAB 10), the whole part of the plant which has grown above the earth and has been collected during flowering, then dried is defined as the medicinal herb [Deutsches Arzneibuch 10, Goldrutenkraut—Solidaginis herba, 1999]. In Hungary, the official medicinal herb is designated as Solidaginis herba that is prepared from the flowery, leafy sprouts of the most often occurring species i.e. Solidago canadensis L. and Solidago gigantea Ait. collected at the beginning of flowering. The quality prescriptions are given in the Hungarian Standard No. 12341-86.

Apáti, P. et al. studied the correlation of phytochemical characteristics and antioxidative properties of classical herbal tea extracts prepared from Canadian goldenrod (Solidago canadensis L.) and determined flavonoids [J. Pharm. Biomed. Analysis, 32, 1045-1053 (2003)]. The authors stated in the introduction that “Canadian goldenrod has been used in European phytotherapy for 700 years for the treatment of chronic nephritis, cystitis, urolithiasis, rheumatism and as an antiphlogistic drug”.

Melzig, M. F. described new mechanisms responsible for the biological effect of goldenrod extracts [Wien. Med. Wochenschr., 154(21-22), 523-527 (2004)]. The herbal preparations prepared from goldenrod extracts are recommended for treatment of infections and inflammations, to prevent formation of kidney stones and to help remove urinary gravel based on a rather complex action spectrum i.e. anti-inflammatory, antimicrobial, diuretic, antispasmodic and analgesic.

Salmond, S. described case studies using phytotherapy, in addition to a huge number of drugs, for curing rather complex symptoms [Aust. J. Med. Herbalism, 14(1), 31-33 (2002)]. One patient suffering from dysuria, glomerulonephritis, benign prostatic hypertrophy, angina pectoris, reflux esophagitis, and, as a side effect of the multiple drug treatment, unstable hypertension and nasal congestion resulting in a daily headache. The daily medication included the administration of 13 different drugs that were completed with a herbal treatment using a mixture of extracts of Solidago virgaurea, Agropyron repens and Althaea off. radix. Two weeks after commencement of treatment there was a slight improvement in reflux esophagitis that remained unchanged after further two weeks. Then, the herbal treatment was completed with the extract of Plantago lanceolata and the dosis was doubled. After further three weeks reflux esophagitis reduced considerably, however, Zantac® (i.e. ranitidine) was still administered, although less frequently. Based on this case study including only one patient it is impossible to state whether the extract of Solidago virgaurea had any favourable effect on the state of the patient. It was likely that the improvement of reflux esophagitis was mainly due to the treatment with the extract of Plantago lanceolata. However, the results cannot be evaluated statistically.

Schmeda-Hirschmann, G. et al. studied the effect of solidagenone, a labdane diterpene occurring in rhizomes of Solidago chilensis Meyen and stated a gastroprotective i.e. antiulcerogenic activity in mice [J. Ethnopharmacology, 81, 111-115 (2002)].

According to Hungarian Patent No. 209 249, an alcoholic extract of Solidago virga-aurea is used together with the extract of other medicinal herbs in a vasodilative preparation. The effect of an extract of Solidago virga-aurea on the blood vessels was studied by Wagner [Wagner, H. H.: Pharmacology of a vasoactive drug containing extract of Solidago, Arzneimittel-Forschung, 16 (7), 859-866 (1966)].

The phytochemical character of various Solidago species shows considerable similarity. The most characteristic active agents thereof include saponins, flavonoids and diterpenes, however, certain species contain significant amounts of ethereal oils, caffeic acid derivatives and simple phenolglycosides. The extracts of Solidago virgaurea L., Solidago canadensis L. and Solidago gigantea Ait. contain, typically, flavonoids, saponins and ethereal oils.

Various editions of Rote Liste and Präparate Liste list several preparations based on a medicinal herb of a Solidago species. The known preparations include e.g. teas, capsules and tablets.

In summary, it can be stated that some of the Solidago species have been already used for the treatment of certain diseases other than the ones related with mitochondrial damage and/or a reduced function of cNOS enzyme.

### SUMMARY OF THE INVENTION

It has been found that the above aim can be achieved by a composition or pharmaceutical composition containing an extract of a part of a Solidago species, wherein said part has grown above the earth, or the solid residue remaining after the removal of the solvent content of the extract as the active ingredient optionally in addition to one or more conventional pharmaceutical carrier(s). The Solidago species belongs to the genus Solidago L. The composition or pharmaceutical composition of the invention is suitable for the enhancement of mitochondrial genesis as well as for the prevention or treatment of diseases deriving from mitochondrial damage and/or reduced function of the constitutive nitric oxide synthase enzyme. The most important diseases of these type have been discussed above.

### DESCRIPTION OF PREFERRED EMBODIMENTS

Under the expression a “Solidago species” mainly the following medicinal plants are meant in terms of the taxonomical description:

### Class: Magnoliopsida

### Subclass: Asteridae

### Family: Asteraceae

### Genus: Solidago L.

### Species:

Solidago alpestris
S. alpicola
S. cambrica
S. canadensis
S. gigantea
S. gigantea ssp. serotina
Solidago graminifolia (L.) Salisb.

### S. Hartmanniana

Solidago x hirtipes Fern

### S. Horvatii

S. jailarum
S. lapponica
S. longifolia
S. macrhorriza
S. maritima
S. minuta
S. monicola

### Solidago x Niederederi

S. scepusiensis
Solidago pauciflosculosa

### S. Pritcheri

S. serotina

### S. Shortii

S. taurica
S. valesiaca
S. virgaurea
S. virgaurea ssp. alpestris
S. virgaurea ssp. macrorrhiza
S. virgaurea ssp. Minuta
S. virgaurea ssp. vulgaris
S. vulgaris
Solidago arguta Ait. ssp. caroliniana (Gray) G. Morton
Solidago arguta Ait. ssp. pseudoyadkinensis G. Morton
Solidago boottii Hook. var. caroliniana (Gray) Cronq.
Solidago yadkinensis (Porter) Small
Solidago arguta Ait. var. harrisii (Steele) Cronq.
Solidago harrisii Steele
Solidago arguta Ait. var. neurolepis (Fern.) Steyermark
Solidago neurolepis Fern.
Solidago x asperula Desf. (pro sp.) [rugosa x sempervirens]
Solidago auriculata Shuttlw. ex Blake
Solidago amplexicaulis Torr. & Gray ex Gray, non Martens
Solidago notabilis Mackenzie
Solidago x beaudryi Boivin [rugosa x uliginosa]
Solidago bicolor L.
Solidago brachyphylla Chapman
Solidago boottii Hook. var. brachyphylla (Chapman) Gray
Solidago buckleyi Torr. & Gray
Solidago caesia L.
Solidago caesia L. var. caesia
Solidago axillaris Pursh
Solidago caesia L. var. axillaris (Pursh) Gray
Solidago caesia L. Var. curtisii (Torr. & Gray) Wood
Solidago caesia L. var. hispida Wood
Solidago curtisii Torr. & Gray
Solidago curtisii Torr. & Gray var. pubens (M. A. Curtis) Gray
Solidago lancifolia Torr. & Gray
Solidago monticola Torr. & Gray
Solidago pubens M. A. Curtis
Solidago calcicola Fern.
Solidago californica Nutt.
Solidago canadensis L.
Solidago canadensis L. var. canadensis
Solidago canadensis L. var. gilvocanescens Rydb.
Solidago altissima L. var. gilvocanescens (Rydb.) Semple
Solidago gilvocanescens (Rydb.) Smyth
Solidago pruinosa Greene
Solidago canadensis L. var. hargeri Fern.
Solidago canadensis L. var. lepida (DC.) Cronq.
Solidago canadensis L. var. subserrata (DC.) Cronq.
Solidago lepida D C.
Solidago lepida D C. var. molina Fern.
Solidago canadensis L. var. salebrosa (Piper) M. E. Jones
Solidago canadensis L. ssp. elongata (Nutt.) Keck
Solidago canadensis L. var. elongata (Nutt.) M. E. Peck
Solidago canadensis L. ssp. salebrosa (Piper) Keck
Solidago dumetorum Lunell
Solidago elongata Nutt.
Solidago lepida D C. var. elongata (Nutt.) Fern.
Solidago lepida D C. var. fallax Fern.
Solidago canadensis L. var. scabra Torr. & Gray
Solidago altissima L.
Solidago altissima L. var. pluricephala M. C. Johnston
Solidago altissima L. var. procera (Ait.) Fern.
Solidago hirsutissima P. Mill.
Solidago lunellii Rydb.
Solidago cutleri Fern.
Solidago deamii Fern.
Solidago discoidea Ell.
Solidago x erskinei Boivin [canadensis x sempervirens]
Solidago fistulosa P. Mill.
Solidago flaccidifolia Small
Solidago graminifolia (L.) Salisb.
Solidago graminifolia (L.) Salisb. var. major (Michx.) Fern.
Solidago x hirtipes Fern.
Solidago graminifolia (L.) Salisb. var. nuttallii (Greene) Fern.
Solidago graminifolia (L.) Salisb. var. polycephala (Fern.) Fern.
Solidago hirtella (Greene) Bush
Solidago nuttallii (Greene) Bush
Solidago polycephala Fern.
Solidago camporum (Greene) A. Nels.
Solidago chrysothamnoides (Greene) Bush
Solidago graminifolia (L.) Salisb. var. gymnospermoides (Greene) Croat
Solidago graminifolia (L.) Salisb. var. media (Greene) S. K. Harris
Solidago gymnospermoides (Greene) Fern.
Solidago gymnospermoides (Greene) Fern. var. callosa S. K. Harris
Solidago media (Greene) Bush
Solidago moseleyi Fern.
Solidago perglabra Friesner
Solidago texensis Friesner
Solidago leptocephala Torr. & Gray
Solidago occidentalis (Nutt.) Torr. & Gray
Solidago galetorum (Greene) Friesner
Solidago graminifolia (L.) Salisb. var. galetorum (Greene) House
Solidago tenuifolia Pursh var. pycnocephala Fern.
Solidago caroliniana B. S. P.
Solidago minor (Michx.) Fern.
Solidago microphylla (Greene) Bush
Solidago microcephala (Greene) Bush
Solidago remota (Greene) Friesner
Solidago tenuifolia Pursh
Solidago sarothrae Pursh
Solidago ptarmicoides (Nees) Boivin
Solidago x bernardii Boivin
Solidago houghtonii Torr. & Gray ex Gray
Solidago x krotkovii Boivin
Solidago x lutescens (Lindl. ex DC.) Boivin
Solidago nitida Torr. & Gray
Solidago ohioensis Frank ex Riddell
Solidago riddellii Frank ex Riddell
Solidago corymbosa Ell.
Solidago jacksonii (Kuntze) Fern.
Solidago rigida L. var. glabrata E. L. Braun
Solidago rigida L. ssp. glabrata (E. L. Braun) Heard & Semple
Solidago rigida L. var. laevicaulis Shinners
Solidago canescens (Rydb.) Friesner
Solidago jacksonii (Kuntze) Fern. var. humilis (Porter) Beaudry
Solidago parvirigida Beaudry
Solidago rigida L. var. humilis Porter
Solidago rigida L. ssp. humilis (Porter) Heard & Semple
Solidago grandiflora Raf.
Solidago rigida
Solidago parryi (Gray) Greene
Solidago graminea (Woot. & Standl.) Blake
Solidago petradoria Blake

### Solidago L.

Solidago albopilosa E. L. Braun
Solidago altiplanities C.& J. Taylor
Solidago puberula Nutt. var. pulverulenta (Nutt.) Chapman
Solidago pulverulenta Nutt.
Solidago pulchra Small
Solidago radula Null.
Solidago radula Nutt. var. laeta (Greene) Fern.
Solidago radula Nutt. var. radula
Solidago pendula Small
Solidago rotundifolia D C.
Solidago scaberrima Torr. & Gray
Solidago radula Nutt. Var. stenolepis Fern.
Solidago roanensis Porter
Solidago maxonii Pollard
Solidago roanensis Porter var. monticola (Torr. & Gray) Fern.
Solidago rugosa P. Mill.
Solidago rugosa P. Mill. ssp. aspera (Ait.) Cronq.
Solidago aspera Ait.
Solidago celtidifolia Small
Solidago drummondii Torr. & Gray
Solidago rugosa P. Mill. var. celtidifolia (Small) Fern.
Solidago rugosa P. Mill. ssp. rugosa
Solidago rugosa P. Mill. ssp. rugosa var. rugosa
Solidago scabra Muhl. ex Willd., non Muhl.
Solidago rugosa P. Mill. ssp. rugosa var. sphagnophila Graves
Solidago aestivalis Bickn.
Solidago rugosa P. Mill. ssp. rugosa var. villosa (Pursh) Fern.
Solidago rupestris Raf.
Solidago canadensis L. var. rupestris (Raf.) Porter
Solidago sciaphila Steele
Solidago sempervirens L.
Solidago sempervirens L. var. mexicana (L.) Fern.
Solidago angustifolia Ell.
Solidago mexicana L.
Solidago petiolata auct. non P. Mill.
Solidago sempervirens L. var. sempervirens
Solidago shortii Torr. & Gray
Solidago simplex Kunth
Solidago simplex Kunth ssp. randii (Porter) Ringius
Solidago simplex Kunth ssp. randii (Porter) Ringius var. gillmanii (Gray) Ringius
Solidago gillmanii (Gray) Steele
Solidago glutinosa Nutt. var. gillmanii (Gray) Cronq.
Solidago racemosa Greene var. gillmanii (Gray) Fern.
Solidago spathulata D C. var. gillmanii (Gray) Gleason
Solidago simplex Kunth ssp. randii (Porter) Ringius var. monticola (Porter) Ringius
Solidago randii (Porter) Britt. var. monticola (Porter) Fern.
Solidago simplex Kunth ssp. randii (Porter) Ringius var. ontarioensis (Ringius) Ringius
Solidago glutinosa Nutt. var. ontarioensis Ringius
Solidago simplex Kunth ssp. randii (Porter) Ringius var. racemosa (Greene) Ringius
Solidago glutinosa Nutt. var. racemosa (Greene) Cronq.
Solidago racemosa Greene
Solidago spathulata D C. var. racemosa (Greene) Gleason
Solidago simplex Kunth ssp. randii (Porter) Ringius var. randii (Porter) Kartesz & Gandhi
Solidago glutinosa Nutt. ssp. randii (Porter) Cronq.
Solidago randii (Porter) Britt.
Solidago spathulata D C. ssp. randii (Porter) Gleason
Solidago simplex Kunth ssp. simplex
Solidago simplex Kunth ssp. simplex var. nana (Gray) Ringius
Solidago bellidifolia Greene
Solidago decumbens Greene
Solidago decumbens Greene var. oreophila (Rydb.) Fern.
Solidago glutinosa Nutt. var. nana (Gray) Cronq.
Solidago oreophila Rydb.

Under “a part of a plant or herb that has grown above the earth” the leaf and/or stem and/or flowers (inflorescence) of the plant is/are meant. Preferably, the extract is prepared from the end of the plant containing many flowers and some leaves. It is especially preferred to prepare the extract from the flowers of the plant.

The extract is prepared in a manner known per se. For this purpose, the part of the plant that has grown above the earth, optionally after drying and size-reducing, is extracted. The extraction is carried out with water or an organic solvent such as an alcohol e.g. ethanol or an aqueous solution of an organic solvent e.g. aqueous ethanol (containing 10-60% by mass of water) generally at 0-100° C., preferably at 20-100° C. During the extraction, in most cases, mixing is applied, however, ultrasonication can be used, too. The extract is separated from the parts of the plant by known methods using e.g. sedimentation, pressing of the parts of the plant, filtration, centrifugation or the combination of the procedures listed. The extract obtained can be used as it is or it can be converted to a liquid composition or pharmaceutical composition such as an aqueous solution or syrup. However, it is preferred to remove the solvent content of the extract for example by evaporation, spray drying or freeze drying (lyophilization), and the solid residue is used as an active agent for the preparation of a composition or a pharmaceutical composition. (In the description and claims, the expression “active agent” is used in this sense and it refers to the solid residue that is dissolved in the extract and can be obtained from the extract of the medicinal herb.) Both the extract and the solid residue obtained from the extract can be characterized by the determination of the flavonoid content. For example, the flavonoid content of the solid residue amounts to 2.7-4.1 g/100 g.

Under a “pharmaceutical composition” a known formulation or dosage form is meant which is conventionally used for the prevention or treatment of diseases and which is suitable for peroral, parenteral, rectal or transdermal administration or for local treatment. Thus, the pharmaceutical composition of the invention is solid or liquid and contains, in addition to the active substance obtained from the medicinal herb by extraction, one or more pharmaceutical carrier(s). The pharmaceutical composition of the invention contains, in general, 0.1-100% by mass, preferably 1-50% by mass, suitably 5-30% by mass of the active ingredient. It is to be noted that a 100% content of active ingredient is possible only in certain cases e.g. in capsules where dilution is not absolutely necessary. In most dosage forms, diluents and/or other auxiliary agents are needed for the preparation of the pharmaceutical composition.

The solid pharmaceutical compositions suitable for peroral administration may be powders, capsules, tablets, film-coated tablets, microcapsules etc., and can comprise binding agents such as gelatine, sorbitol, poly(vinylpyrrolidone) etc.; filling agents such as lactose, glucose, starch, calcium phosphate etc.; auxiliary substances for tabletting such as magnesium stearate, talc, poly(ethylene glycol), silica etc.; wetting agents such as sodium laurylsulfate etc. as the carrier.

The liquid pharmaceutical compositions suitable for peroral administration may be solutions, suspensions or emulsions and can comprise e.g. suspending agents such as gelatine, carboxymethylcellulose etc.; emulsifiers such as sorbitane monooleate etc.; solvents such as water, oils, glycerol, propylene glycol, ethanol etc.; preservatives such as methyl or propyl p-hydroxybenzoate etc. as the carrier.

Pharmaceutical compositions suitable for parenteral application contain, in general, a sterile solution of the active agent.

Pharmaceutical compositions suitable for local treatment include solutions, creams, liniments etc.

The dosage forms listed above as well as other dosage forms are known per se, see e.g. the manual Remington's Pharmaceutical Sciences, 18th edition, Mack Publishing Co., Easton, USA (1990)

The pharmaceutical composition contains dosage unit, in general. The daily dose can be administered in one or more portions. The actual dosage depends on many factors and is determined by the doctor. In general, a typical dose for adult patients of 70 kg body weight amounts to 0.1 to 10 g, preferably 1 to 5 g of active agent, daily.

In general, the pharmaceutical composition is prepared by admixing the active ingredient to one or more carrier(s) and transforming the mixture obtained into a pharmaceutical composition in a manner known per se. The methods that can be used are known from the literature e.g. the manual Remington's Pharmaceutical Sciences cited above. Of course, as a further possibility, the solid residue obtained from the extract can be directly filled into capsules or the extract itself can be converted to a liquid pharmaceutical composition by the addition of further carriers, if needed.

In cases when the extract or active ingredient of the invention is applied to achieve mitochondrial genesis for e.g. roboration, muscle-building etc. purpose, the composition administered is not necessarily a pharmaceutical composition, however, the contents and preparation thereof corresponds to those of the pharmaceutical compositions described herein. Consequently, the expression “composition” used in the description and claims without the marking “pharmaceutical” refers to a dosage form that is applied to induce favourable biological effects related to mitochondrial genesis, however, such treatment is not definitely medication.

The effect of the Solidago species was studied by the following biological tests.

Cell Cultures and Cultural Characteristics Employed in the In Vitro Tests

Primary Pig Endothelial Cell Culture

The thoracic aorta of a pig was excised, aseptically, and placed into a PBS solution containing 50 μg/ml of streptomycin for transport (PBS is a physiological saline that contains phosphate buffer). The connective tissue was removed from the aorta which latter was cut to pieces of several cm length, and the rings obtained were opened. A sterile, close-meshed plastic net impregnated with trypsin (0.25% of trypsin in PBS) was placed onto the surface covered by endothelium and the tissues were kept at 37° C. for 1 or 2 minute(s). The endothelium layer loosened under the action of trypsin was removed by washing, the cells were collected by centrifugation, then removed to culturing dishes coated with collagen and grown in a 1:1 mixture of DMEM (Dulbecco's modified Eagle's medium) culture medium (GibcoBRL, Eggenstein, Germany) supplemented with 10% of FCS (fetal calf serum) and F-12. A homogenous culture was obtained in which more than 95% of the cells showed endothelial morphology. The cells from the first ten passage were used in the tests.

Primary Rat Glia Culture

The cortex of an 8 day's old Wistar rat was removed aseptically, cut into pieces, and the cells were set free by digestion with trypsin (0.08% of trypsin in PBS). The larger pieces of tissue were removed, the fraction containing individual cells and lumps consisting of some cells was washed twice with RPMI (Roswell Park Memorial Institute) culture medium containing 10% of FCS. At last, the cells suspended in RPMI culture medium containing 10% of FCS were placed into grow dishes (Greiner) without special surface treatment and grown at 37° C. under an atmosphere containing 5% of carbon dioxide. A mixed cell culture was obtained containing mainly glia (astrocyte, oligodendrocyte and microglia) cells. Cells obtained in the first passage were used in the tests.

HaCaT Cell Culture

Human immortalized HaCaT skin cells were grown in a DMEM culture medium containing 10% of FCS in dishes (Greiner) without special surface treatment at 37° C. under an atmosphere containing 5% of carbon dioxide. The culture medium was supplemented with 25 mM or 50 mM of D-glucose and the cells were grown for at least 1 week in this culture medium.

Quantitative Determination and Morphological Study of Mitochondria

Staining with the Fluorescent Stain MitoTracker

The cells were incubated with 100 nM of the fluorescent stain MitoTracker at 37° C. for 30 minutes. The stain cumulating in the active mitochondria exhibits a fluorescent emission at 516 nm following an excitation at 490 nm. The fluorescence of the mitochondria was determined partly in a microscope, partly using a FACS (fluorescence activated cell sorter) apparatus. For the determination with the microscope, a fluorescence microscope Zeiss-Axioskop was employed. Exposures were prepared with a Nicon Coolpix 995 digital camera using identical exposure time, diaphragm aperture, digital picture size and optical enlargement. The exposures were evaluated by densitometry. The intensity of the cell fluorescence is proportional to the amount of mitochondria. In addition, the observation under a microscope allows the study of the morphology of the mitochondrial network. The FACS analysis was carried out using a Becton Dickinson FACS Calibur apparatus.

Staining with the Fluorescent Stain JC-1

The cells were incubated on a culture medium containing 1 μM of JC-1 fluorescent stain (Molecular Probes) at 37° C. for 30 minutes. The JC-1 stain acumulates in the active mitochondria depending on the mitochondrial membrane potential. In case of high membrane potential, the JC-1 stain forms aggregates in the mitochondrion while in case of low membrane potential, the monomeric form is typical. The monomeric and aggregate form of JC-1 stain have different emission peak (at 530 nm and 590 nm, respectively) following an excitation at 490 nm. The mitochondrial membrane potential was determined by means of Olympus BX-51 fluorescent microscope and Cell Analysis Software.

Determination of ATP

The cells were rinsed and collected in an ice-cold PBS solution (pH=7.4) containing 5 mM of ethylenediaminetetra-acetic acid (EDTA), 5 mM of sodium fluoride and 100 μM of Na₃VO₄. The cell pellet was lyzed in a solution containing 0.5% of trichloroacetic acid over ice. The insoluble cell debris was removed by centrifugation (13000 g, 5 minutes, +4° C.). The amount of ATP in the clear supernatant was determined by means of an ATP Determination Kit (Molecular Probes). The luminescence was measured with a WALLAC 1450 microbeta Plus apparatus.

Determination of a Mitochondrion Specific Protein by Means of Western Blot

The cells were rinsed and collected in ice-cold PBS (pH=7.4) containing 5 mM of EDTA, 5 mM of sodium fluoride and 100 μM of Na₃VO₄. Lysis of the cell pellet was carried out on ice under weak shaking for 10 minutes in a buffer solution containing 250 mM of sodium chloride, 50 mM of HEPES [4-(2-hydroxyethyl)-1-piperazine-ethanesulfonic acid] (pH=7.4), 1 mM of EDTA, 1 mM of EGTA [ethyleneglycol-bis(2-aminoethyl ether)-N,N,N′,N′-tetraacetic acid], 1.5 mM of magnesium chloride, 0.1% of Nonidet P-40 [nonylphenyl-poly(ethylene glycol)], 40 mM of β-glycerol phosphate, 1 mM of Na₃VO₄, 1 mM of phenylmethylsulfonyl fluoride, 10 mM of benzamidine, 20 mM of sodium fluoride, 10 mM of sodium pyrophosphate, 10 μM/ml of aprotinin, 10 μg/ml of leupeptin, and 10 μg/ml of antipain. The insoluble cell debris was removed by centrifugation (13000 g, 12 minutes, +4° C.). The clear supernatant was admixed to 1/2 volume of 2× Laemmli gel loading buffer, the samples were boiled for 3 minutes, then maintained at −20° C. before use. The protein concentration was determined by means of Bio-Rad D_{c} Protein Assay reagent (Bio-Rad Laboratories, Hercules, Calif., USA). The protein samples were separated by polyacrylamide gel electrophoresis in the presence of 10% of sodium dodecyl-sulfate (10% SDS-PAGE) and blotted on PVDF [poly-(vinylidene difluoride)] membrane using a Trans-Blot SD Blotting Kit (Bio-Rad Laboratories). The imune detection was carried out using the following antibodies: anti-COX-IV antibody (A21348, Molecular Probes), anti-HSP72 (Citomarker Research & Development, Hungary), anti-HSP90 alpha (Affinity BioReagents, Golden, USA), anti-eNOS (Transduction Laboratories, USA) and anti-HSP60 (Affinity BioReagents, Golden, USA). For the detection, ECL (enhanced chemi-luminescence) Plus System (Amersham) was employed.

The evaluation of the microscope exposures and Western blots was carried out densitometrically by means of a UTHSCA Image Tool Version 3.0 computer program. In case of the microscope exposures, three confluent cell layers selected randomly were examined. From the optical density values obtained, the mean value was determined. Statistical comparison and calculation were carried out by the one-way analysis of the difference using the Posthoc Newman-Keuls test (Pharmacological Calculation System). The statistical significance was p≦0.05.

Study of the Intestinal Motility in Rats

The test was carried out on rat ileum preparations according to Anjaneyulu [Anjaneyulu, M. and Pamarao, P.: Studies on gastrointestinal tract functional changes in diabetic animals, Methods Find. Exp. Clin. Pharmacol., 24, 71-75 (2002)]. In male Sprague-Dowley rats (Charles River Hungary) of 230-260 g body mass, diabetes was induced by the intravenous administration of 60 mg/kg of streptozocin. The blood sugar level of the animals was determined from the fourth week following the induction of diabetes, weekly, and the animals having stable high blood sugar level (>20 mM/litre) were drawn into the test after the tenth week. The 7 animals of the test group were treated orally, once daily, with a dose of 30 mg/kg of the active substance prepared from Solidago canadensis according to Example 1, process A for 5 days, while the 7 animals of the control group were treated with an identical volume of physiological saline. On the day after the last treatment, the animals were anaesthetized by the administration of pentobarbital, the ileum was removed, cleaned, suspended in a Krebs-Henseleit's solution at 37° C. using 1 g of initial load, contracted with acetylcholine, then the relaxation ability of the ileum was evaluated based on the relaxation response to encreasing doses of isoproterenol [4-[1-hydroxy-2-[(1-methylethyl)amino]-ethyl]-1,2-benzenediol]. The measurements were carried out in an Isosys System apparatus (Experimetria, Budapest, Hungary).

The following results were obtained in the tests:

Increase of the Mitochondrial Number

In general, a culture medium having a higher glucose content (25 mM) than the normal blood sugar level is used for growing human immortalized HaCaT keratinocyte cells. In the culture medium, the cells adapted themseves to the high glucose concentration. Therefore, in order to simulate the hyperglycaemic environment, the concentration of glucose was raised by further 25 mM and the cells were grown in this culture medium. Based on staining with MitoTracker, the mitochondrion content of the cells grown in the culture medium containing 25 mM of glucose was higher by 30% than that of the cells grown under hyperglycaemic circumstances in a culture medium containing 50 mM of glucose. This observation corresponds to the known fact that a hyperglycaemic environment deteriorates mitochondria. The cell culture pretreated with 50 mM of glucose was treated for 4 days with a dose of 8 μg/ml of the active substance prepared from the medicinal herb Solidago canadensis by extraction and lyophilization of the extract according to Example 1, process A. The treatment raised the amount of mitochondria by 45% i.e. the treatment could combat the hyperglycaemic effect and enhanced the mitochondrial number, significantly.

An increase of the mitochondrial number due to the treatment with an aqueous solution containing 8 μg/ml of an active substance prepared from the medicinal herb Solidago canadensis according to Example 1, process A for 4 days was observed in primary pig endothelial cell culture. After staining with the fluorescent stain Mito-Tracker, the results were determined using a fluorescence activated cell sorter (FACS) apparatus. In the evaluation of the results obtained, the optical density of the control cell culture was taken as unit and the optical densities of the treated cell cultures were compared with that of the control cell culture. Evaluation with the FACS apparatus indicated an increase of the mitochondrial number by 380%.

The increase of mitochondrial number owing to the treatment for 4 days with a dose of 8 μg/ml of the active substance obtained from the medicinal herb Solidago canadensis according to Example 1, process A was shown in primary pig endothelial cell culture using JC-1 stain, too. Since the monomeric JC-1 accumulates in the mitochondria, the increase of the amount thereof indicates the increase of the mitochondrial number. Only a very low amount of JC-1 aggregate representing a high membrane potential could be detected. The treatment with the active substance extracted from Solidago canadensis raised the amount of mitochondria by a factor of 5 compared with the control. The strengthening of the mitochondrial network could be observed after the treatment on the microscope exposures.

The ATP content of cell is closely related to the state and membrane potential of mitochondria. The determination of ATP in primary pig endothelial cells cultured under normoglycaemic (in the presence of 10 mM of glucose) and hyperglycaemic (in the presence of 30 mM of glucose) environment indicated an increase of the ATP content by a factor of 4.7 and 5.3, respectively, compared with the untreated control cells.

In a primary rat glia cell culture, the treatment carried out with a dose of 16 μg/ml of the active substance prepared from the medicinal herb Solidago canadensis according to Example 1, process A for 4 days resulted in a 100% increase of the fluorescence characterizing the amount of mitochondria compared with the control. The strengthening of the mitochondrial network could be observed after the treatment in this case, too.

In addition to the direct determination of the mitochondrial number, an increase of the level of COX-IV protein could be also noticed on Western blot. It is to be noted that the COX-IV protein is specific of mitochondrion and plays a key role in oxidative phosphorylation, thus, the increase of the level thereof indicates the increase of the amount of mitochondria. It was found that the COX-IV protein level increased by a factor of 6 compared with the control in the primary rat glia cell culture owing to the treatment for 4 days with a dose of 16 μg/ml of the active substance prepared from the medicinal herb Solidago canadensis according to Example 1, process A.

Increase of the Expression of cNOS and HSP

Nitric oxide synthetized especially by the endothelial nitric oxide synthase (eNOS) enzyme is rather important in the regulation of mitochondrial function and biogenesis. Nitric oxide enhances the expression of the transcription factor PGC-1α, the main regulator of mitochondrial biogenesis. The heat shock proteins (chaperons) HSP72 and HSP90 are extremely important in the formation and stabilization of the functionally active eNOS complex. It is to be noted that, in addition to the stabilization of the cNOS enzyme system, the HSP72 has a key role also in the import of mitochondrial proteins. This coordinated import of protein is essential for the normal mitochondrial function and biogenesis.

The proteins examined by us and having a key role in mitochondrial biogenesis were not expressed or only a very low amount of them were expressed in the HaCaT keratinocyte cells cultured in hyperglycaemic environment. However, treatments for 4 days with doses of 8, 16 or 32 μg/ml of the active substance extracted from the medicinal herb Solidago canadensis according to Example 1, process A raised the amount of the proteins examined considerably as shown in Table 1 based on staining with Mito-Tracker and determination by Western blot. Correspondingly, the mitochondrial network could be detected once more in hyperglycaemic cells after the treatment.

TABLE 1
Increase of the amount of proteins in the cells

Relative optical density

| | | Concentration of Solidago | | |
|---|---|---|---|---|
| | | canadensis | | |
| Protein | Control | 8 μg/ml | 16 μg/ml | 32 μg/ml |
| e-NOS | ND | 1.5 | 3.2 | 5.9 |
| PGC-1α | ND | 2.0 | 4.0 | 8.0 |
| HSP-90α | ND | 3.0 | 4.5 | 4.7 |
| HSP-72 | ND | 3.0 | 3.8 | 4.2 |
| HSP-60 | 1.0 | 10.0 | 8.25 | 6.6 |
| COX-IV | 1.0 | 3.0 | 2.0 | 2.0 |

ND = not detectable, the value is well below 1.0.

From Table 1 it can be seen that four proteins that are essential for mitochondrial function and biogenesis could have not been detected in the control group, however, owing to the treatment of the invention, after 4 days, the amount thereof has already been considerable. The amount of heat shock protein HSP-60 increased by 6.6-10 times, that of protein COX-IV increased by 2-3 times owing to the treatments of the invention.

Toxicity Test

5 male NMRI white mice having a body mass of 23-25 g were treated, once, with a dose of 200 mg/kg of the active substance of Solidago canadensis prepared according to Example 1, process A, intraperitoneally. The behaviour of the animals was evaluated for a week: neither any change of behaviour, nor weight loss was experienced. Thus, it can be stated that a single i.p. dose of 200 mg/kg of the active substance extracted from the medicinal herb Solidago canadensis does not result in an acute toxic effect in mice.

Restoration of Reduced Intestine Relaxation

The intestine motility test described above which was carried out on the ileum isolated from artificially diabetic rats gave the results summarized in Table 2.

TABLE 2

| | Relaxation of ileum induced |
|---|---|
| | with 10⁻⁷ M of isoproterenol in |
| | % of maximum relaxation |
| Control rats | 54.1 ± 3.2 |
| Rats treated with streptozocin | 33.2 ± 2.4 |
| Rats treated with streptozocin | 52.8 ± 3.1 |
| and a p.o. dose of 20 mg/kg of | |
| the active substance extracted | |
| from Solidago canadensis | |

The data of Table 2 show that the contraction developed with acetylcholin is considerably compensated by isoproterenol on the ileum of healthy animals in the control group. Treatment with streptozocin reduced the relaxation significantly, however, treatment with the active substance extracted from Solidago canadensis restored the relaxation ability of the ileum.

The above in vitro and in vivo tests prove that an extract of a part of a Solidago species, wherein said part has grown above the earth, or the solid residue remaining after the removal of the solvent content of the extract as the active ingredient enhances the mitochondrial genesis and compensates the reduced function of the constitutive nitric oxide synthase enzyme. The effect on mitochondrial genesis becomes pronounced when the usual mitochondrial content of the cells is reduced by a pathological effect e.g. hyper-glycaemia. In the background of the increase of mitochondrial genesis, an enhanced expression and function of chaperon and cNOS proteins were observed. Nitric oxide that depends on cNOS stimulates mitochondrial biogenesis and increases the expression of transcription factors that regulate biogenesis. The chaperons (HSP70, HSP90, HSP60, HSP27) partly contribute to the formation and stabilization of the functionally active cNOS complex, partly have an important role in the transport of mitochondrial proteins as well as in the compensation of any oxidative load (e.g. hyperglycaemia).

Therefore, it is expected that an extract of a part of a Solidago species, wherein said part has grown above the earth, or the solid residue remaining after the removal of the solvent content of the extract as the active ingredient provides for a curing effect in case of disorders owing to a damage of the mitochondrion or a reduced function of the cNOS enzyme; advantages can be awaited in states or diseases when an increase of mitochondrial genesis is useful; furthermore, diseases connected with a damage of the mitochondrion or the reduced function of the cNOS enyzme can be prevented by applying said extract or active ingredient.

An extract of a part of a Solidago species, wherein said part has grown above the earth, or the solid residue remaining after the removal of the solvent content of the extract as the active ingredient can be effective especially in the following states:

I. Through exerting an influence on the mitochondrial function and promoting the mitochondrial biogenesis
a) States and diseases requiring fast mitochondrial regeneration:
long-lasting immobilization, states following a disease accompanied by weight loss,
regeneration phase of anorexia.
   b) States requiring increased mitochondrial demand:
muscle (especially striated muscle) developing training,
sudden loading of the muscles (especially striated muscles) and the subsequent period,
muscular strain
adaptation to high-altitude.
   c) Neurodegenerative diseases:
ALS (amyotrophic lateral sclerosis),
Huntington's disease,
Alzheimer's disease,
Parkinson's disease.
   II. Other application possibilities due to improvement of cNOS function:
   a) Motility disorders of the gastrointestinal system:
achalasia,
infantile hypertrophic pylorus stenosis,
Hirschprung's disease,
diabetic gastropathy,
reflux oesophagitis,
gastrointestinal function disorder in case of diabetes,
gastroparesis,
functional dyspepsia,
intestinal pseudoobstruction and colitis,
common motility disorders of the gastrointestinal system (e.g. obstipation),
dysfunction of sphincters (e.g. pyloric sphincter, sphincters of the anus).
   b) Gall bladder dysfunctions:
biliary dyskinesia,
formation of gallstone,
dyslipidemia,
types II and III biliary and pancreatic sorts of sphincter of Oddi dysfunction (SOD),
post-cholecystectomy syndrome,

Thus, the invention refers to the use of an extract of a part of a Solidago species, wherein said part has grown above the earth, or the solid residue remaining after the removal of the solvent content of the extract as the active ingredient for the preparation of a composition useful in the regeneration of the organism of a mammal after long-lasting immobilization, anorexia, states following a disease or accompanied by weight loss as well as for muscle development or muscle growth during muscle developing training, treatment of muscular strain and adaptation to high-altitude.

A preferred composition is a roborant composition that improves the physical condition of the body after illness or anorexia or in case of muscle development trainings.

Furthermore, the invention refers to the use of an extract of a part of a Solidago species, wherein said part has grown above the earth, or the solid residue remaining after the removal of the solvent content of the extract as the active ingredient for the preparation of a pharmaceutical composition suitable for the prevention or treatment of neurodegenerative diseases and/or motility disorders of the gastrointestinal system.

A preferred embodiment of the invention refers to the use of an extract of a part of a Solidago species, wherein said part has grown above the earth, or the solid residue remaining after the removal of the solvent content of the extract as the active ingredient for the preparation of a pharmaceutical composition suitable for the prevention or treatment of neurodegenerative diseases comprising ALS, Parkinson's disease, Alzheimer's disease and Atkinson's disease.

Another preferred embodiment of the invention refers to the use of an extract of a part of a Solidago species, wherein said part has grown above the earth, or the solid residue remaining after the removal of the solvent content of the extract as the active ingredient for the preparation of a pharmaceutical composition suitable for the prevention or treatment of motility disorders of the gastrointestinal system comprising achalasia, infantile hypertrophic pylorus stenosis, Hirschprung's disease, diabetic gastropathy, reflux oesophagitis, gastrointestinal function disorder in case of diabetes, gastroparesis, functional dyspepsia, intestinal pseudoobstruction, colitis, common motility disorders of the gastrointestinal system and dysfunction of sphincters.

An especially preferred embodiment of the invention refers to the use of an extract of a part of a Solidago species, wherein said part has grown above the earth, or the solid residue remaining after the removal of the solvent content of the extract as the active ingredient for the preparation of a pharmaceutical composition suitable for the prevention or treatment of motility disorders of the gastrointestinal system, thus, the pharmaceutical composition is a prokinetic agent that induces movement in the gastrointestinal system.

According to a still preferred embodiment of the invention, the motility disorder of the gastrointestinal system is a dysfunction of sphincters.

The invention includes a method for regeneration of the organism of a mammal after long-lasting immobilization, anorexia, states following a disease or accompanied by weight loss as well as for muscle development or muscle growth during muscle developing training, treatment of muscular strain and adaptation to high-altitude in which the mammal being in need thereof is treated with a therapeutically effective amount of an extract of a part of a Solidago species, wherein said part has grown above the earth, or the solid residue remaining after the removal of the solvent content of the extract as the active ingredient.

Furthermore, the invention includes a method for the prevention or treatment of neurodegenerative diseases and/or motility disorders of the gastrointestinal system, in which the patient being in need thereof is treated with a therapeutically effective amount of an extract of a part of a Solidago species, wherein said part has grown above the earth, or the solid residue remaining after the removal of the solvent content of the extract as the active ingredient.

The invention is further elucidated by means of the following Examples.

### Example 1

### Preparation of an Extract

Process A

100 g of the dry, finely powdered parts of Solidago canadensis grown over the earth and comprising mainly flowers are extracted with water in a mass ratio of 5:200 at 60° C. under intensive stirring over a water bath. The aqueous extract obtained is filtered, the plant matter is pressed, then the extract is sedimented for 4-8 hours, and filtered again. The dry matter content of the aqueous extract obtained amounts to 6.2-6.9 mg/ml. The water is removed by lyophilization while maintaining the temperature of the tray under −50° C. The dry residue obtained is stored in darkness at room temperature and protected from moisture. The dry matter (i.e. the active substance) has a flavonoid content of 3.1-3.4 g/100 g.

Process B

100 g of the dry, powdered flowers of Solidago canadensis are extracted with water in a mass ratio of 5:150 by boiling at 100° C. The aqueous extract obtained is worked up as described under process A. The aqueous extract has a dry matter content of 8.5-9.1 mg/ml. The lyophilized product (i.e. active substance) prepared as given under process A has a flavonoid content of 3.8-4.1 g/100 g.

Process C

100 g of the dry, powdered parts of Solidago canadensis grown over the earth (i.e. leaf, stem, flowers) are extracted with aqueous ethanol containing 75% by volume of ethanol in a mass ratio of 5:200 in a cold ultrasonic bath. The extract is filtered and the ethanol is removed by evaporation under reduced pressure. The remaining aqueous phase is dried by lyophilization as described under process A.

### Example 2

### Preparation of Capsules

0.6 g portions of the lyophilized active substance prepared according to Example 1, process B are filled into hard gelatin capsules, the capsules are closed, placed into a glass container that is sealed airtightly.

### Example 3

### Preparation of Syrup

To 1000 ml of the aqueous extract prepared according to Example 1, process A (dry matter content: 6.2 mg/ml), 20 ml of glycerol, 100 ml of 70% aqueous sorbitol solution, 0.1 g of aroma substance and 1 g of methyl paraben are added, the mixture is homogenized and filled into bottles of 50 ml.

1. Use of an extract of a part of a Solidago species, wherein said part has grown above the earth, or the solid residue remaining after the removal of the solvent content of the extract as the active ingredient for the preparation of a composition useful in the regeneration of the organism of a mammal after long-lasting immobilization, anorexia, states following a disease or accompanied by weight loss as well as for muscle development or muscle growth during muscle developing training, treatment of muscular strain and adaptation to high-altitude.

2. Use of an extract of a part of a Solidago species, wherein said part has grown above the earth, or the solid residue remaining after the removal of the solvent content of the extract as the active ingredient for the preparation of a pharmaceutical composition suitable for the prevention or treatment of neurodegenerative diseases and/or motility disorders of the gastrointestinal system.

3. A use according to claim 2 in which the motility disorder of the gastrointestinal system is a dysfunction of sphincters.

4. A method for regeneration of the organism of a mammal after long-lasting immobilization, anorexia, states following a disease or accompanied by weight loss as well as for muscle development or muscle growth during muscle developing training, treatment of muscular strain and adaptation to high-altitude in which the mammal being in need thereof is treated with a therapeutically effective amount of an extract of a part of a Solidago species, wherein said part has grown above the earth, or the solid residue remaining after the removal of the solvent content of the extract as the active ingredient.

5. A method for the prevention or treatment of neurodegenerative diseases and/or motility disorders of the gastrointestinal system, in which the patient being in need thereof is treated with a therapeutically effective amount of an extract of a part of a Solidago species, wherein said part has grown above the earth, or the solid residue remaining after the removal of the solvent content of the extract as the active ingredient.
